(19) United States
(12) Patent Application Publication
(10) Pub. No.: US 2014/0180248 A1
(43) Pub. Date: Jun. 26, 2014

(54) Osmotic Ablation Device

(71) Applicant: Erez Salik, Westport, CT (US)

(72) Inventor: Erez Salik, Westport, CT (US)

(21) Appl. No.: 13/726,130

(22) Filed: Dec. 23, 2012

(51) Int. Cl. A61M 25/10

(52) U.S. Cl. CPC A61M 25/10
USPC 604/509; 604/103.01; 604/506; 604/101.05; 604/99.04

(57) ABSTRACT

A tissue ablation device comprising a balloon catheter, or other expandable element, wherein said expandable element is comprised of a semipermeable membrane. The balloon membrane is expanded by an osmotically active solution to contact a vascular endothelium, or other desired tissue, whereupon the resultant osmotic gradient affects a water flux from the vascular endothelium, or other desired tissue, across the semipermeable membrane and towards the osmotically active solution.

### CROSS-REFERENCE TO RELATED APPLICATIONS

This application claims priority to provisional application No. 61/580,261 filed Dec. 26, 2011.

### STATEMENT REGARDING FEDERALLY SPONSORED RESEARCH OR DEVELOPMENT

Not Applicable

### FIELD OF THE INVENTION

The present invention is in the technical field of medical devices. More particularly, the present invention relates to tissue ablation, in particular vascular ablation by chemical means.

### BACKGROUND OF THE INVENTION

Lower extremity chronic venous disorder (CVD) is a medical condition affecting up to 50% of all Americans. The spectrum of CVD ranges from varicose veins and telangiectasias, which can be symptomatic and unsightly, to severe edema, skin ulceration and ultimately major disability. This condition is most often caused by venous hypertension induced by incompetent valves in the superficial veins of the leg. Incompetent valves, which lead to venous hypertension, most commonly occur in the great saphenous vein (GSV), but can also occur in the small saphenous vein (SSV), and in any of a multitude of perforator veins which communicate between the deep and superficial venous systems of the leg.

The traditional therapy for treatment of varicose veins dates back to the ancient Greek, who described a method of surgical ligation and stripping of varicose veins. Surgical ligation and stripping remained the mainstay of varicose vein treatment until the more recent introduction of endovascular techniques for saphenous vein ablation. These techniques, which utilize laser or radiofrequency energy delivered through endovascular techniques, have been shown to result in shorter recovery time, fewer complications, and more durable results when compared to traditional ligation and stripping. Current treatments for CVD resulting from superficial venous insufficiency include surgical ligation and stripping, injection sclerotherapy, and endovenous ablation using laser or radiofrequency energy, or any combination of the above.

Although the introduction of endovascular techniques represented a significant advancement in the treatment of venous insufficiency, there are several shortcomings. The energy deposited by the laser or radiofrequency probe, although effective at ablating the target vein, has the potential to damage adjacent structures including nerves, muscle, fat and overlying skin. To minimize this risk, most practitioners use tumescent anesthesia—a lidocaine and saline solution deposited around the target vein that acts as an insulator between the ablation catheter and surrounding structures. Most often the tumescent anesthesia is delivered through a needle which is advanced under ultrasound guidance, and most often requires multiple needle punctures. This process is not only painful and time consuming, but also introduces the risk of lidocaine toxicity secondary to inadvertent intravenous injection, and infection secondary to multiple punctures.

Injection sclerotherapy is another non-surgical alternative to surgical stripping. Sclerotherapy involves the injection of a sclerosing agent which results in chemical ablation of the venous endothelium. Sclerosants such as hypertonic saline cause dehydration and cell membrane denaturation along the venous endothelium. Sclerosants such as polidocanol and sodium tetradecyl sulfate are week detergents that disrupt the endothelial cellular membrane. Delivery of these sclerosants, which are typically injected as a liquid, is inherently inaccurate. Excessive injection of sclerosant can cause the solution to propagate into the deep venous system, and therefore increases the risk of deep vein thrombosis; conversely, insufficient volume will fail to sclerose the target vessel. In addition, extravasation of sclerosant can be painful and potentially damaging to surrounding tissue.

A device which would allow for precise, reliable tissue ablation without threat of injury to surrounding tissue would represent a significant improvement in the state of the art for the treatment of venous insufficiency, as well as for tissue ablation elsewhere in the body.

### SUMMARY OF THE INVENTION

Forward osmosis is a process that uses a semi-permeable membrane to effect separation of water from dissolved solutes. The driving force for this separation is an osmotic pressure gradient, such that a “draw” solution of high concentration (relative to that of the “feed” solution), is used to induce a net flow of water through the membrane into the draw solution, thus effectively separating the feed water from its solutes. The simplest equation describing the relationship between osmotic and hydraulic pressures and water flux is: Jw=A(Δπ−ΔP) where Jw is water flux, A is the hydraulic permeability of the membrane, Δπ is the difference in osmotic pressures on the two sides of the membrane, and ΔP is the difference in hydrostatic pressure (negative values of Jw indicating reverse osmotic flow).

The present invention describes a balloon catheter, or other catheter with an expandable element along the distal aspect, where the expandable element is comprised in part or in whole of a semipermeable membrane. The balloon is insufflated with a hyperosmolar (relative to cytoplasm) draw solution until the balloon contacts the tissue to be ablated, for example the vessel endothelium. The osmotic pressure gradient between the intracellular cytoplasm and balloon lumen results in flux of water across the cell membrane and semipermeable membrane of the balloon, into the balloon lumen. This would result in cellular dehydration and cell membrane desiccation, similar to that induced by intravascular injection of hypertonic saline. Any crystalloid or colloid hyperosmolar solution, or any hygroscopic material, that would affect the flow of water across the membrane may be used.

In addition to the permeability characteristics of the membrane, the balloon would ideally, but not necessarily, be constructed from a highly compliant material that would keep the internal pressure of the balloon low, and thereby facilitate forward osmosis across the balloon. A compliant balloon would also allow for improved apposition between the balloon and target tissue along tortuous segments and varying diameters of the vessel. Alternatively, a non-compliant material could be used if it provides more favorable permeability characteristics, with the geometry of the balloon or balloons altered to improve tissue apposition and water flux across the membrane.

Although the invention is discussed with specific reference to venous ablation in the treatment of venous insufficiency, the invention is contemplated to have wider applications. For example (but not limited to), the device can be used to achieve sclerosis of the gonadal vein in the treatment of varicocele or pelvic congestion syndrome, sclerosis of fallopian tubes in lieu of tubal ligation, ablation of the uterine endometrium for the treatment of dysfunctional uterine bleeding, or in sclerosis of pulmonary veins in the treatment of arrhythmias or pulmonary arteriovenous malformations. In addition, the device can be used to induce osmotic damage to renal sympathetic nerves as a potential treatment for resistant hypertension. The properties of the balloon membrane and insufflating solution, as well as the balloon geometry, could be altered in accordance with need. For example, the balloon can be filled with a hypotonic solution, to allow flux of fluid out of the balloon.

### BRIEF DESCRIPTION OF THE DRAWINGS:

FIG. 1A illustrates a cross section of the balloon catheter in the deflated state.

FIG. 1B illustrates a cross section of the balloon catheter in the inflated state.

FIG. 1C illustrates a cross section of the balloon catheter formed to include a guidewire lumen.

FIG. 2A illustrates a cross sectional view of the catheter in FIGS. 1A and 1B taken along line A.

FIG. 2B illustrates a cross sectional view of the catheter in FIG. 1C taken along line A.

FIG. 3 illustrates a balloon catheter inflated within a blood vessel.

FIG. 4A illustrates a cross section of an alternative embodiment of the balloon catheter incorporating an inflow and outflow lumen.

FIG. 4B illustrates a cross section of an alternative embodiment of the balloon catheter that incorporates an inflow and outflow lumen, as well as a guidewire lumen.

FIG. 5A illustrates a cross sectional view of the catheter in FIG. 4A taken along line B.

FIG. 5B illustrates a cross sectional view of the catheter in FIG. 4B taken along line B.

FIG. 6 illustrates a cross sectional view of an alternative embodiment of the balloon catheter where the balloon is spiraled along the central shaft.

FIG. 7 illustrates a cross sectional view of an alternative embodiment of the balloon catheter where several compartments are created within the balloon lumen.

FIG. 8 illustrates a cross sectional view of an alternative embodiment of the balloon catheter where the balloon is comprised of lobes along the shaft.

FIG. 9A illustrates an alternative embodiment of the balloon catheter where the balloon contains several elongated lobes oriented along the long axis of the shaft.

FIG. 9B illustrates a cross sectional view of the catheter in FIG. 9A taken along line C.

### DETAILED DESCRIPTION OF THE INVENTION

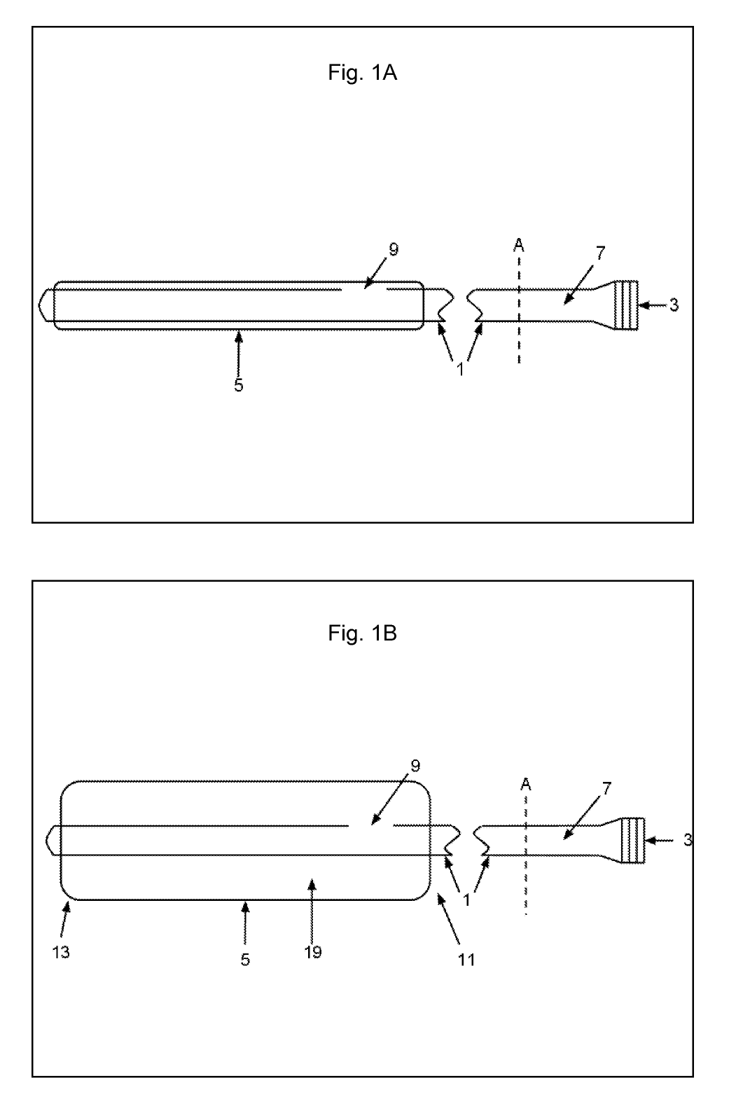

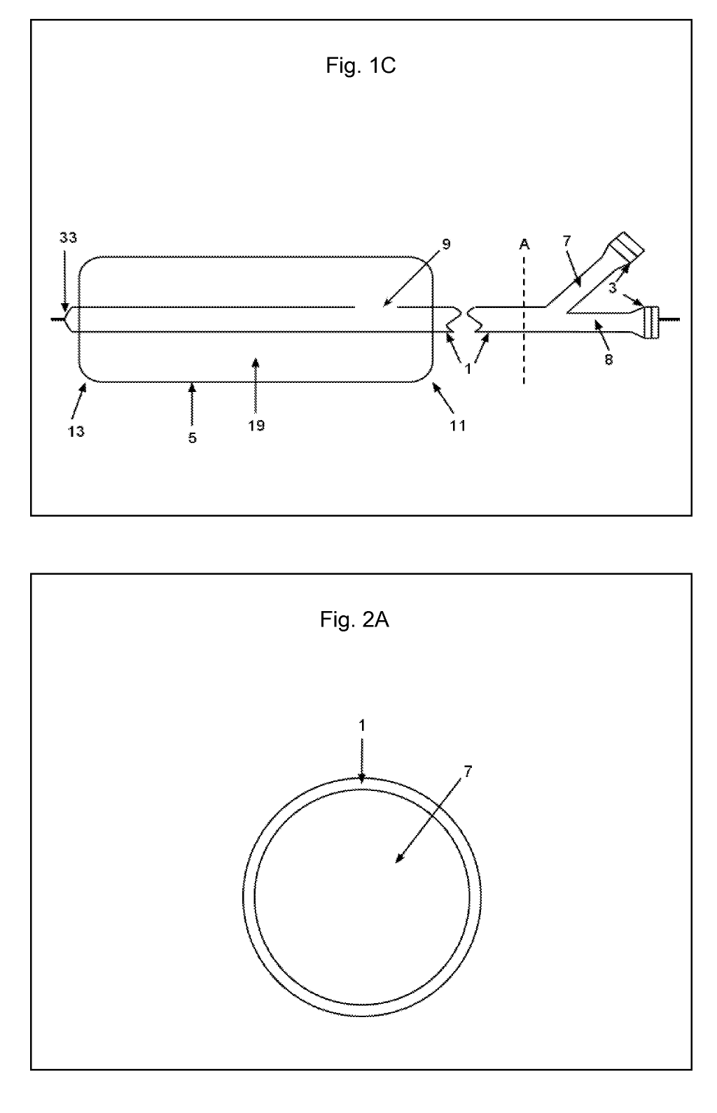

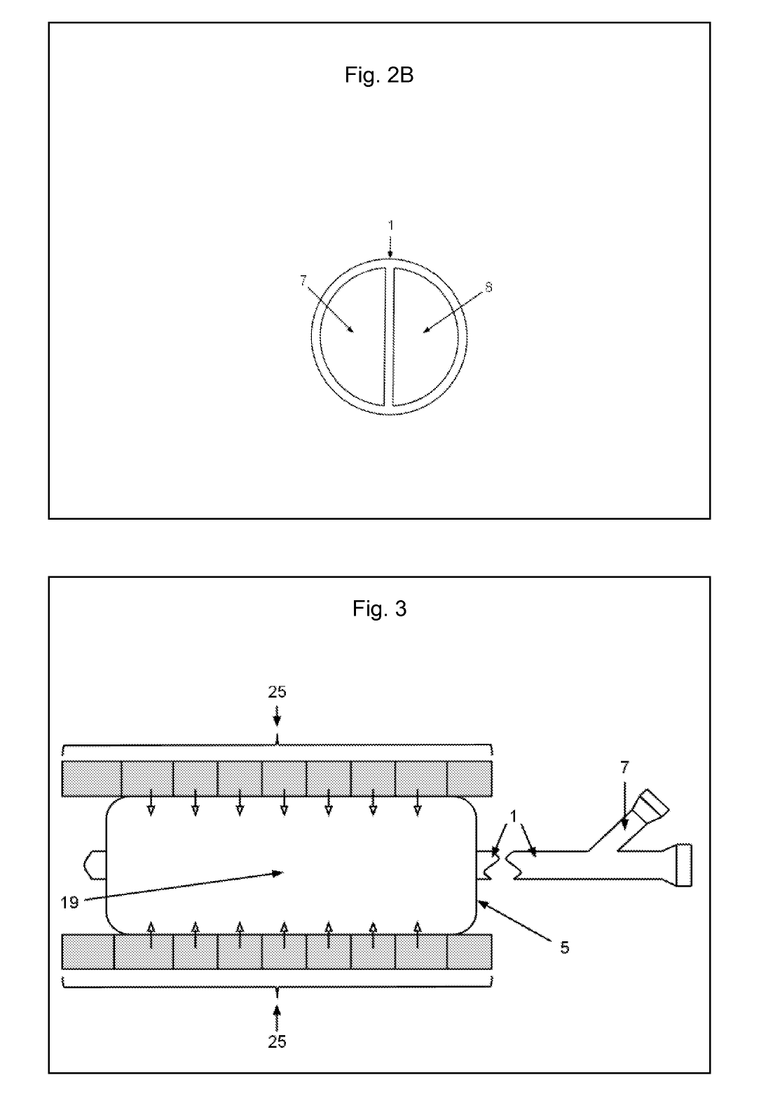

An embodiment of the present invention is illustrated in FIG. 1A, FIGS. 1B and 1C, and in cross section in FIGS. 2A and 2B. The catheter includes an elongated flexible shaft (1) with a proximal end and a distal end. An inflatable and deflatable balloon (5) is mounted on the distal end of elongated shaft (1). The catheter shaft (1) includes an inflation lumen (7) that extends from the proximal end of the shaft and terminates in an opening (9). It may be beneficial to have the balloon inflate from its proximal pole to its distal pole, such that blood (or other fluid) is displaced distally as the balloon expands, thereby minimizing the potential for trapped blood (or other fluid) and improving apposition between the balloon (5) and target tissue. Therefore, the opening (9) is preferably positioned at the proximal end of the balloon (5) to promote expansion of the balloon from its proximal end (11) to its distal end (13). The proximal end of the catheter shaft (1) carries a fitting (3) by which the inflation lumen (7) may be connected to a syringe or other fluid delivery device that allows inflation of the balloon with a draw solution. As illustrated in 1C and 2B, the catheter shaft (1) may be formed to include a guidewire lumen of desired diameter (8) that extends through the catheter and terminates in an outlet orifice (33) at the distal end of the catheter.

FIG. 3 illustrates the balloon (5) in a potential application, in this example expanded within a blood vessel. The balloon is expanded with a hypertonic (>300 mOsm/L) draw solution such that the balloon contacts the vessel endothelium (25). The osmotic pressure within the balloon is substantially higher than the intracellular osmotic pressure. This gradient effects flux of water (open arrows) out of the endothelial cells (25), across the semipermeable membrane of the balloon (5), and into the balloon lumen (19).

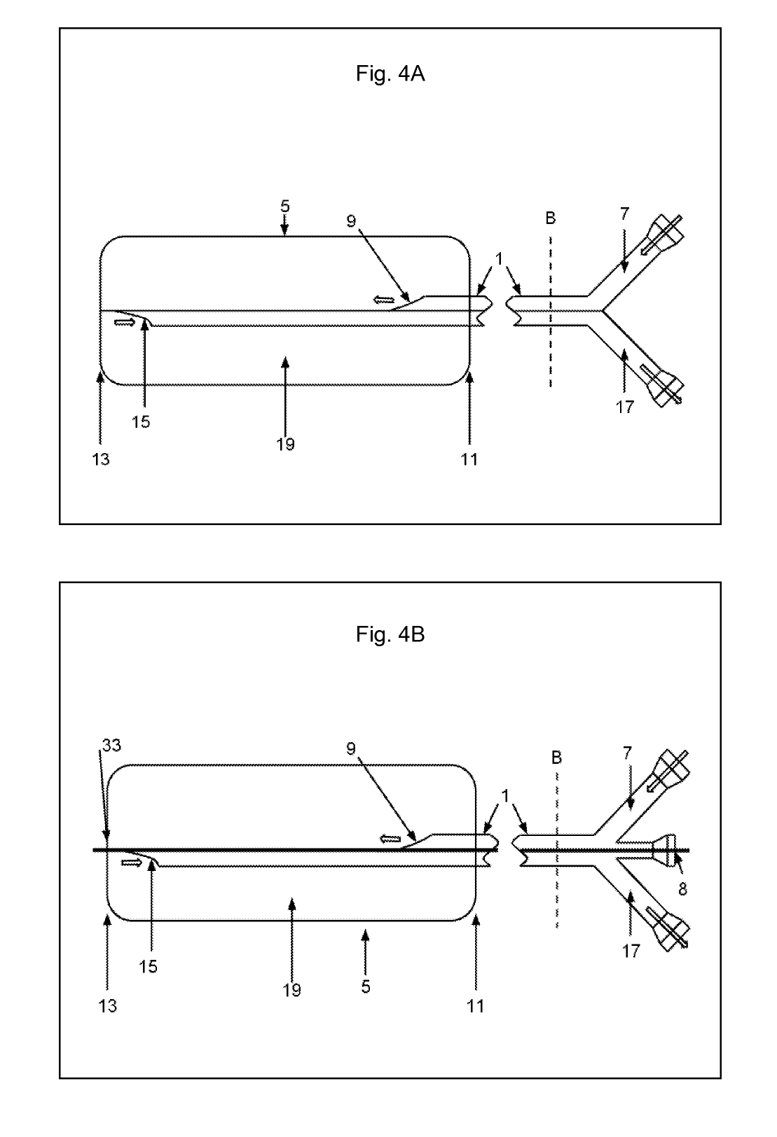

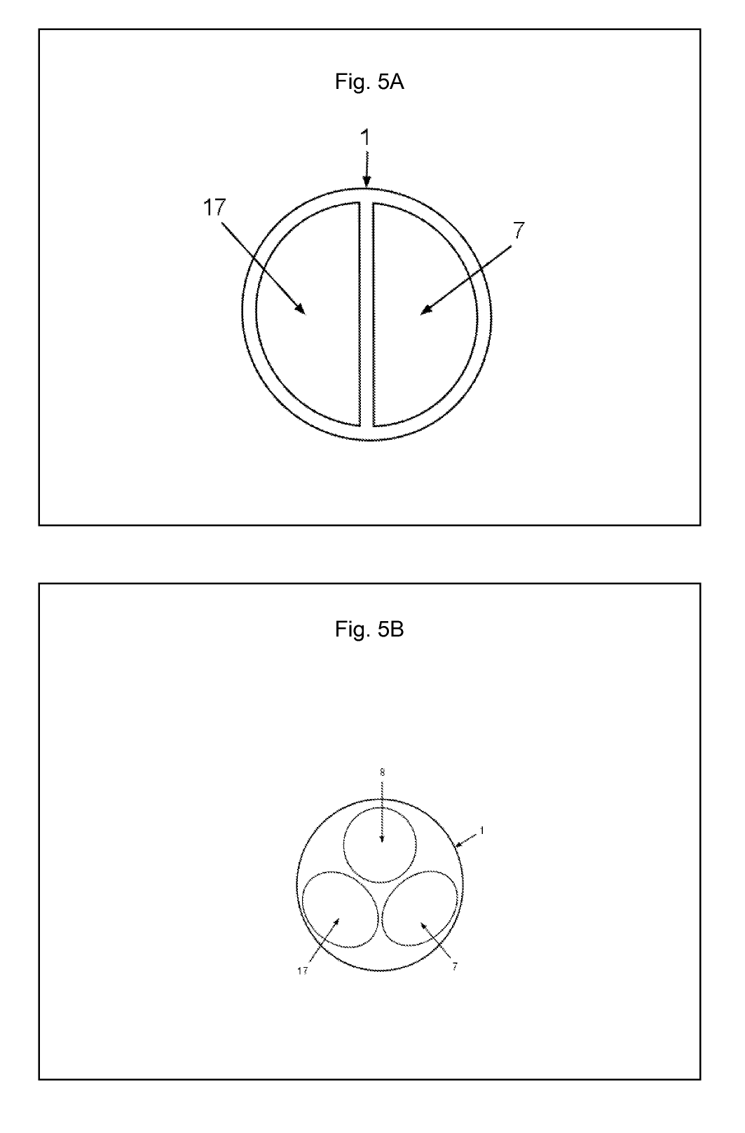

As illustrated in FIGS. 4A and 4B, the catheter shaft (1) may be formed to include an outflow lumen (17) which communicates with the balloon lumen (19) through an opening (15) on the shaft (1), and terminates in a secondary port at the proximal end of the catheter. In the embodiment of FIGS. 4A and 4B, the outflow lumen (15) is positioned at the distal end of the balloon (5). The outflow lumen (15) would allow for circulation of fluid within the balloon lumen, (19), thereby maintaining a uniform concentration of the draw solution within the balloon. Maintaining a uniform concentration will decrease dilutive concentration polarization within the balloon, and would facilitate flux of water across the semipermeable balloon membrane. Since increased hydrostatic pressure within the balloon would inhibit water flux across the balloon membrane, the outflow lumen may be fitted with a pressure sensitive valve (not pictured) to prevent over-pressurization of the balloon. As illustrated in FIGS. 5A and 5B, the catheter shaft (1) may be formed without (FIG. 5A) or with (FIG. 5B) a guidewire lumen (8).

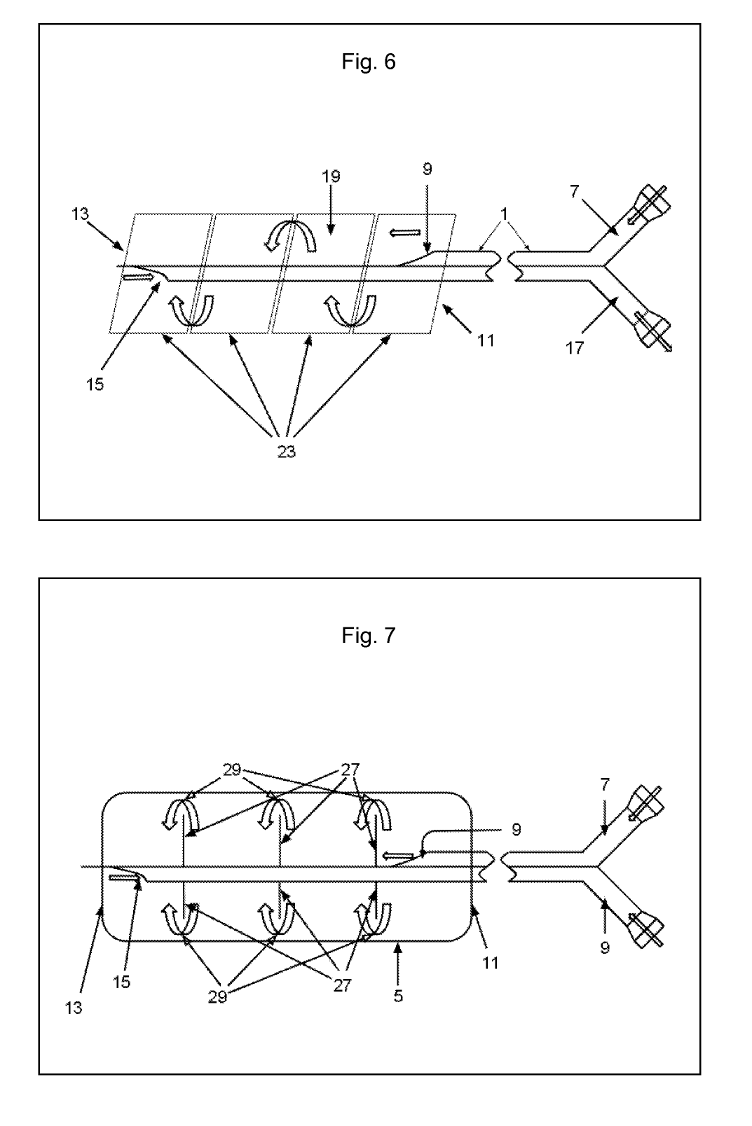

FIG. 6 illustrates an alternative embodiment which may further improve circulation of the draw solution within the balloon lumen, and further facilitate displacement of blood (or other fluid) from the lumen of the target vein (or other target tissue). The balloon, rather than being longitudinally mounted on the catheter shaft (1), is wound around the shaft (1) in a spiral (23). The draw solution is infused through the inflow lumen (7), with the opening to the lumen (9) placed near the proximal pole of the balloon (11), expanding the spiraled balloon from the proximal pole (11) to the distal pole (13). An opening (15) for the outflow lumen (17) is situated near the distal pole of the balloon (13), to promote circulation of draw solution within the balloon lumen (19). This design may also improve contact between the balloon and the vessel wall, thereby improving water flux from the endothelium (25) into the balloon lumen (19).

FIG. 7 illustrates an alternative embodiment where the balloon is longitudinally mounted on the shaft (1), though the balloon contains multiple septa (27) which define several compartments within the balloon (5). The septae are fenestrated (29), to allow for flow between the segments (curved arrows). This promotes sequential filling of the balloon from the proximal pole (11) to the distal pole (13), and facilitate displacement of fluid from the vascular lumen.

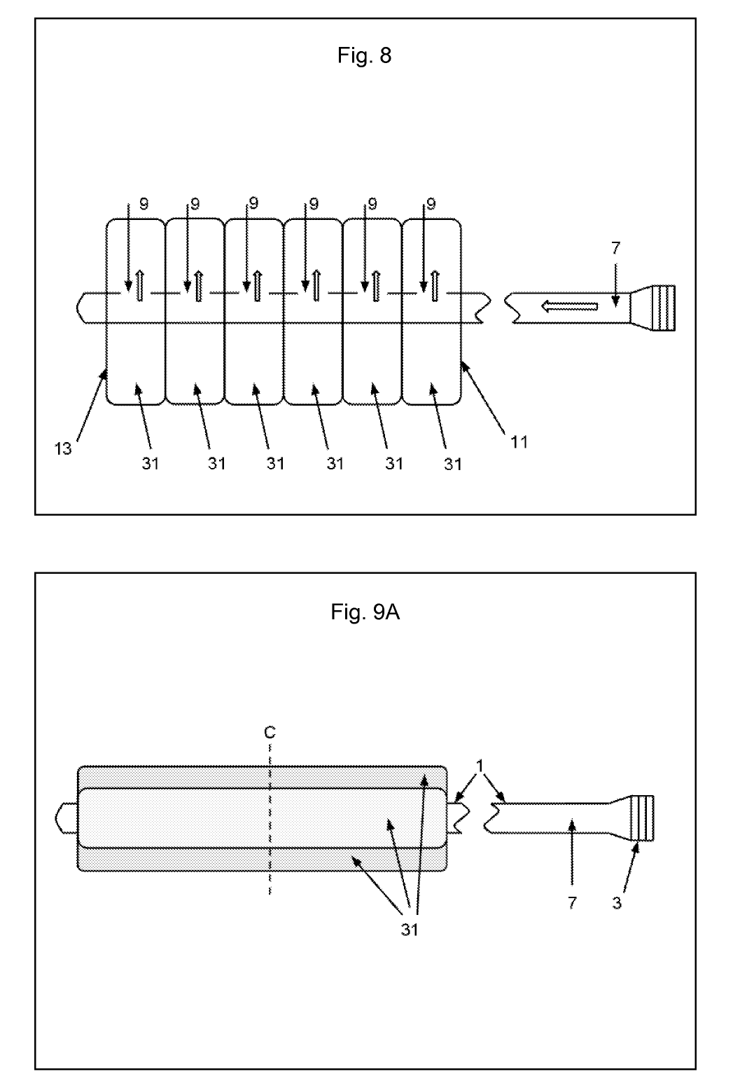

FIG. 8 illustrates an alternative embodiment where the balloon contains separate lobes (31), each lobe having an independent opening (9) on the catheter shaft (1) in communication with the inflation lumen (7). The intervening septa between the lobes (31) do not contain fenestrations. The lobes (31) are arranged in series along the shaft (1), and each lobe (31) communicates with the inflation lumen (7) through a separate opening (9). When the draw solution is infused through the inflow lumen, the lobes inflate in series, from the proximal lobe (11) to the distal lobe (13).

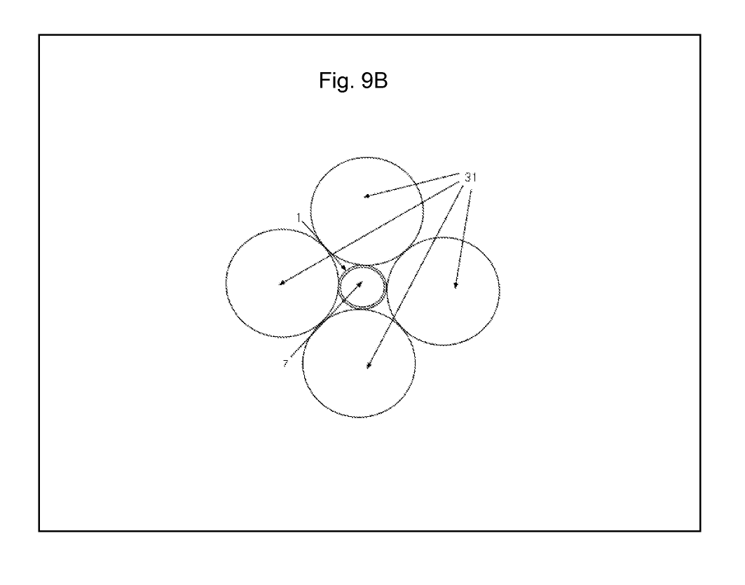

FIGS. 9A and 9B illustrates an alternative embodiment where the balloon contains several elongated lobes (31) oriented along the long axis of the shaft (1). In this embodiment four lobes of equal size are depicted, thereby giving a 4-leaf clover appearance on cross section (FIG. 9B). The number of lobes and the size of the individual lobes can be increased or reduced to promote apposition between the balloon membrane and the vessel wall, and to promote diffusion across the balloon membrane. The lumena of individual lobes may be in communication to allow circulation of fluid between the lobes. Alternatively, the lumen of any or all lobes may be isolated from the others, with separate inflow openings (9) for any or all lobes (31). In addition, and outflow port (15) and lumen (17) can be incorporated into any or all lobes (31).

While the foregoing written description of the invention enables one of ordinary skill to make use what is considered presently to be the best mode thereof, those of ordinary skill will understand and appreciate the existence of variations, combinations, and equivalents of the specific embodiment, method and examples herein. The invention should therefore not be limited by the above described embodiments, methods and/or examples, but by all embodiments and methods within the scope and spirit of the invention as claimed.

I claim:

1. A tissue ablation device comprising:
a balloon catheter, wherein said balloon catheter is comprised of a semipermeable membrane;
wherein said balloon catheter being insufflated with an osmotically active solution.

2. A tissue ablation device comprising:
an elongated shaft having at least one lumen therethrough, a proximal end portion and distal end portion, the proximal and distal end portions defining a longitudinal axis there between;
an expandable element positioned on the distal end of said shaft, with said expandable element defining at least one chamber which is in fluid communication with one or more of the said lumen, and which when expanded is in substantial direct contact with tissue to be ablated;
a fluid material injected into said expandable element with said material being osmotically active and generally hyperosmolar relative to body tissue, and said fluid material giving rise to an osmotic pressure gradient which could affect water flux from body tissue to fluid material;
a semipermeable membrane comprising a portion or the entirety of said expandable element, with said membrane permeable to water and impermeable to the osmotically active agent in said fluid material, such that upon substantial direct contact with the tissue to be ablated, said membrane promotes an osmotically driven water flux from tissue, through said membrane, and into said chamber;

3. The apparatus of claim 2 wherein said expandable element is expanded by an osmotically active crystalloid or colloid solution.

4. The apparatus of claim 2 wherein said expandable element is expanded by a hygroscopic material.

5. The apparatus of claim 2 wherein expandable element is a single balloon defining a single chamber.

6. The apparatus of claim 2 wherein expandable element comprises two or more elongated balloons oriented such that the longitudinal axis of said elongated balloons parallels said longitudinal axis of shaft, with each of said balloons in fluid communication with at least one lumen of said shaft.

7. The apparatus of claim 5 wherein each of said balloons is equal in length and diameter.

8. The apparatus of claim 5 wherein said balloons are of variable length and diameter.

9. The apparatus of claim 2 wherein said expandable element is a balloon defining multiple chambers wherein each of the said chambers is in fluid communication with at least one additional chamber, and at least one of the said chambers is in fluid communication with at least one lumen of said shaft.

10. The apparatus of claim 2 wherein said expandable element comprises two or more balloons placed in series along the distal end of said shaft, with each balloon defining one or more chamber which is in fluid communication with at least one lumen on said shaft.

11. The apparatus of claim 2 wherein said expandable element is a balloon which is substantially spiraled along said shaft.

12. The apparatus of claim 2 wherein said semipermeable membrane is a compliant material.

13. The apparatus of claim 2 wherein said semipermeable membrane is a non-compliant material.

14. The apparatus of claim 2 wherein said expandable element includes a hygroscopic compound situated within, around, or alongside said membrane.

15. The apparatus of claim 2 with a single lumen through shaft allowing for delivery of said liquid material into said chamber defined by said expandable element.

16. The apparatus of claim 2 wherein said shaft accommodates at least one inflow lumen for delivery of said liquid material into said chamber, and at least one separate outflow lumen, also in fluid communication with said chamber, and allowing withdrawal of said liquid material from said chamber.

17. The apparatus of claim 15 wherein said inflow lumen is in fluid communication with said chamber at a point on said shaft more proximal than said outflow lumen.

18. The apparatus of claim 15 wherein a pressure sensitive valve is fitted along the proximal end of said shaft, in fluid communication with said outflow lumen.

19. The apparatus of claim 15 wherein said fluid material is circulated within said chamber.

20. The apparatus of claim 2 wherein an additional lumen is configured to receive a guidewire.

21. A method for osmotically induced destruction of body tissue, said method comprising:
advancing an elongated catheter into said tissue, wherein said catheter has an expandable element at the distal end comprised in part or in whole of a semipermeable membrane;
instillation of an osmotically active liquid material into said expandable element and bringing said expandable element into substantial contact with said tissue;
affecting a water flux from said tissue, through said semipermeable membrane, and into chamber defined by said expandable element;
wherein the osmotically driven water flux results in tissue desiccation and destruction;

22. The method according to claim 21 wherein there is no direct contact between sclerosant and body tissue.

23. The method according to claim 21 wherein a pressure limiting valve prevents pressure build up within said expandable element.

24. The method according to claim 21 wherein said liquid material is circulated within the chamber defined by said expandable element.

25. The method according to claim 24 wherein separate inflow and outflow ports allow for circulation of said liquid material.

26. The method according to claim 21 wherein said expandable element expands from its proximal aspect on said shaft to its distal aspect.

27. The method according to claim 21 wherein said expandable element expands from its distal aspect on said shaft to its proximal aspect.

28. The method according to claim 21 wherein said body tissue is a vascular structure.

29. The method according to claim 21 wherein said body tissue is a Fallopian tube.

30. The method according to claim 21 wherein said body tissue is the endometrium of the uterus.

31. The method according to claim 21 wherein said body tissue is a peri-vascular neural plexus.

32. The method according to claim 21 whereby tissue destruction is achieved without the need for tumescent anesthetic.

33. A method for body tissue sclerosis wherein sclerosis is achieved without direct contact of the sclerosant and said body tissue.
